(19) United States
(12) Patent Application Publication
(10) Pub. No.: US 2011/0286881 A1
(43) Pub. Date: Nov. 24, 2011

(54) Methods for controlling and reducing pathogens, allergens odor-causing agents

(76) Inventors: James L. Marsden, Manhattan, KS (US); Joseph P. Urso, Dallas, TX (US)

(21) Appl. No.: 12/801,029

(22) Filed: May 18, 2010

(51) Int. Cl. A61L 9/015; A61L 2/18

(52) U.S. Cl. 422/5; 422/28

(57) ABSTRACT

Disclosed are methods for reducing and/or preventing infection and controlling and/or reducing the level of one or more pathogens, allergens and/or odor-causing agents.

### FIELD OF THE INVENTION

The present invention relates to methods for controlling and/or reducing the level of one or more pathogens, allergens and/or odor-causing agents.

### BACKGROUND OF THE INVENTION

Pathogens, allergens and odor-causing agents, including (but not limited to) pathogenic microbes, molds, mildew, spores, and organic and inorganic pollutants, are commonly found in a wide range of environments. At a minimum, these substances can cause discomfort and, at the other extreme, serious illness and death.

Microbial control and disinfection in environmental spaces is therefore generally desirable to improve health, and an absolute necessity in medical spaces, food processing spaces and the like. Numerous ways have been used to in the past in an attempt to purify air and disinfect surfaces.

For example, it is known that Reactive Oxidizing Species (“ROS”), such as those produced by, e.g., photocatalytic oxidation processes, can oxidize organic pollutants and kill microorganisms. More particularly, the products of photocatalytic reactions, such as hydroxyl radicals, hydroperoxyl radicals, chlorine and ozone, are known to be capable of oxidizing organic compounds and killing microorganisms. There are, however, limitations to the methods and devices known and available to those skilled in the art, not only because of efficacy limitations but also because of potential safety issues.

“ROS” is a term used to describe the highly activated air that results from exposure of ambient humid air to ultraviolet light. Light in the ultraviolet range (i.e. 10-400 nm) emits photons at a frequency that has sufficient energy to break chemical bonds when absorbed. UV light at wavelengths of 250-255 nm is routinely used as a biocide. Light below about 181 nm, up to 182-187 nm, is competitive with corona discharge in its ability to produce ozone.

UV radiation is currently being used for disinfection in community water systems, as is ozone. Ozone is also currently being used to treat industrial wastewater and cooling towers.

Hydrogen peroxide is similarly known to have antimicrobial properties and has been used in aqueous solution for disinfection and microbial control.

Many prior attempts to use hydrogen peroxide in the gas or vapor phase, however, have been hampered by technical hurdles associated with the desire to produce “purified” hydrogen peroxide. More particularly, vaporized aqueous solutions of hydrogen peroxide generally produce an aerosol of microdroplets composed of aqueous hydrogen peroxide solution.

Various processes for “drying” vaporized hydrogen peroxide solutions produced, at best, a hydrated form of hydrogen peroxide. This was believed undesirable since the hydrated hydrogen peroxide molecules were surrounded by water molecules bonded by electrostatic attraction and/or London Forces. It was further believed that the ability of the hydrogen peroxide molecules to directly interact with the environment by electrostatic means was greatly attenuated by the bonded molecular water. Accordingly, past efforts were directed at reducing or eliminating the water molecules bonded to the hydrogen peroxide.

Moreover, the lowest concentrations of vaporized hydrogen peroxide that could be achieved were generally well above the 1.0 ppm OSHA workplace safety, limit, making these processes unsuitable for use in occupied areas.

Photocatalysts have been used in the past to reduce or eliminate organic pollutants in fluid. Such photocatalysts include, but are not limited to, TiO2, ZnO, SnO2, WO3, CdS, ZrO2, Sb2O4 and Fe2O3. Of these, titanium dioxide (TiO2) is chemically stable, has a suitable bandgap for UV/Visible photoactivation, and is relatively inexpensive. The photocatalytic chemistry of titanium dioxide has therefore been the object of extensive studied over the last thirty years for its ability to reduce or eliminate organic and/or inorganic compounds from contaminated air and water.

Because photocatalysts can generate hydroxyl radicals from absorbed water when activated by UV light of sufficient energy, they have been used to produce hydrogen peroxide gas for release into the environment. Prior uses of photocatalysis, however, have primarily focused on the generation of a plasma containing many different reactive chemical species. Further, the majority of the chemical species in the photocatalytic plasma are reactive with hydrogen peroxide, and therefore inhibit the production of hydrogen peroxide gas by means of reactions that destroy hydrogen peroxide. Also, any organic gases that are introduced into the plasma inhibit hydrogen peroxide production both by direct reaction with hydrogen peroxide and by reaction of their oxidized products with hydrogen peroxide.

The photocatalytic reactor itself also limits the production of vaporized hydrogen peroxide for release into the environment. Because hydrogen peroxide has greater chemical potential than oxygen to be reduced as a sacrificial oxidant, it is preferentially reduced as it moves downstream in photocatalytic reactors as rapidly as it is produced by the oxidation of water.

### Oxidation:

2 photons+2H₂O→2OH.+2H++2e−2OH.→H₂O₂

### Reduction:

2OH.+2H++2e−→2H₂O

Additionally, several side reactions generate a variety of species that become part of the photocatalytic plasma and inhibit the production of hydrogen peroxide gas for release into the environment.

The wavelengths of light used to activate photocatalysts are also energetic enough to photolyze the peroxide bond in a hydrogen peroxide molecule and are also an inhibitor in the production of vaporized hydrogen peroxide for release into the environment. Further, the practice of using wavelengths of light that produce ozone introduces yet another species into the photocatalytic plasma that destroys hydrogen peroxide.

O₃.H₂O₂.→H₂O+2O₂

In practice, the use of photocatalysts has focused on the production of a plasma, often containing ozone, which is then used to oxidize organic contaminants and microbes. Since these plasmas are primarily effective within the confines of the reactor itself, these devices are designed to pass air through the reaction chamber for disinfection. See, for example, U.S. Pat. No. 6,955,791. As such, they are of limited use in disinfecting either large spaces or specific objects.

Such plasmas also have limited chemical stability beyond the confines of the reactor and actively degrade hydrogen peroxide gas.

Further, because the plasma is only really effective within the reactor itself, many designs actually try maximize residence time within the reactor to try to facilitate more complete oxidation of organic contaminants and microbes as they pass through the reactor. Since hydrogen peroxide has such a high potential to be reduced, this maximized residence time concomitantly results in minimized hydrogen peroxide production.

Also, most uses of photocatalysts produce environmentally objectionable chemical species. First among these is ozone itself, an intentional product of many systems. Ozone, however, is potentially harmful and levels thereof are strictly regulated.

Moreover, since organic contaminants that pass through a reactor are seldom oxidized in a single exposure, multiple air exchanges may be necessary to achieve full oxidation to carbon dioxide and water. As incomplete oxidation occurs, various aldehydes, alcohols, carboxylic acids, ketones, and other partially oxidized organic species can be produced in the reactor. Often, photocatalytic reactors can actually increase the overall concentration of organic contaminants in the air by fractioning large organic molecules into multiple small organic molecules, such as formaldehyde.

Other prior attempts to use ozone and hydrogen peroxide for disinfection have sought to control the temperature and humidity of the environment being cleaned. For example, U.S. Pat. No. 7,407,624 discloses a method for abating allergens, pathogens, odors and volatile compounds in a sealed enclosure using specific concentrations of ozone and hydrogen peroxide in an environment at a specific temperature and specific humidity. This method, however, is not practical because of the need to seal the environment being disinfected and the levels of ozone are far too high for human safety.

There therefore remains a need in the art for an effective method for controlling and/or reducing the level of one or more pathogens, allergens and/or odor-causing agents.

### SUMMARY OF THE INVENTION

It is therefore an object of the present invention to provide for methods for controlling and/or reducing the level of one or more pathogens, allergens and/or odor-causing agents using combinations of ozone and hydrogen peroxide generated in a humid environment.

In accordance with this and other objects, a first embodiment of the present invention is directed to a method for controlling and/or reducing the level of a substance selected from the group consisting of a pathogen, an allergen and an odor-causing agent, comprising: exposing the substance to a gaseous atmosphere comprising an effective amount of ozone and hydrogen peroxide, wherein the hydrogen peroxide is generated in an environment having a relative humidity of at least 70%. The substance is exposed to this gaseous atmosphere for a time period sufficient to abate the substance(s) being targeted.

Other embodiments of the present invention are directed to devices for generating a gaseous atmosphere comprising an effective amount of ozone and hydrogen peroxide, wherein the hydrogen peroxide is generated in an environment having a relative humidity of at least 70%.

It is to be understood that both the foregoing general description and the following detailed description are exemplary and explanatory only and are intended to provide further explanation of the invention as claimed.

### DETAILED DESCRIPTION OF PREFERRED EMBODIMENTS

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

Preferred embodiment of the present invention include methods for controlling and/or reducing the level of a substance selected from the group consisting of a pathogen, an allergen and an odor-causing agent, comprising: exposing the substance to a gaseous atmosphere comprising an effective amount of ozone and hydrogen peroxide, wherein the hydrogen peroxide is generated in an environment having a relative humidity of at least 70%. The substance is exposed to this gaseous atmosphere for a time period sufficient to abate the substance(s) being targeted.

The methods of the preferred embodiments use mixtures of ozone and hydrogen peroxide vapor, each within particular concentration range. Significantly, the hydrogen peroxide vapor used in the inventive methods is generated in an environment having a particular humidity. This process yields a gaseous atmosphere having high concentrations of hydrogen peroxide, and low (but still effective) concentrations of ozone.

By utilising the gaseous atmospheres of the present invention, a prescribed concentration of ozone and hydrogen peroxide vapor can be applied to a target material, such as an object or surface, for a prescribed period of time, thereby resulting in the control and/or reduction of most odors, bacteria, viruses, molds, allergens, and the like.

Once application is completed, the device(s) used to generate the hydrogen peroxide vapor and ozone may be removed from the treated area and the air within ventilated to the outside, such as through open doors and windows. Alternatively the hydrogen peroxide vapor and ozone may be left to naturally decompose back to oxygen and water. Given that the half life of ozone is measured in minutes under the conditions employed in the inventive methods and that the half life of hydrogen peroxide is very short when in the presence of ozone, the inventive methods are generally safe to employ in inhabited areas.

Ozone has a long history of use in purification methods. The Food and Drug Administration approved ozone for use in food preservation in June 2001. Currently, ozone is used to purify most of the tap and bottled water in the US, as well as keeping fruits and vegetables fresh during storage. Low powered ozone generators have been commercially available for many years for in-home use. These units, however, have proven to be very problematic.

More specifically, prior units that were safe to operate with people present generally generated levels of ozone that were believed far too low to be effective in abating mold and other pathogens and allergens. Conversely, more powerful units that were effective in decontaminating would frequently generate levels of ozone that were unsafe for continuous occupation by animals or people.

The concentrations of ozone used in the methods of preferred embodiments, however, are generally safe for animals and people. Thus, unlike prior methods, during use of the methods of the present invention, animals and people do not need to remain outside an area being treated. Moreover, following treatment, ozone levels in the area treated can be quickly reduced to that of the outside environment and no potentially harmful residual substances are left behind.

According to particularly preferred embodiments of the present invention, the concentration of ozone is generally less than 0.0070 ppm (parts per million). More preferably, the concentration of ozone is less than 0.0067 ppm, even more preferably less than 0.0065 ppm and still even more preferably less than 0.0063 ppm. According to yet other preferred embodiments, the concentration of ozone is less than 0.0068 ppm, more preferably less than 0.0066 ppm, even more preferably less than 0.0064 ppm and still even more preferably less than 0.0062 ppm. Most preferably, the concentration of ozone employed in the methods of the present invention is less than 0.0060 ppm.

The hydroxyl radical has been found to be significantly more reactive than direct oxidation by the ozone molecule. Hydroxyl radicals can be produced by the reaction between ozone and water.

The amount of hydroxyl radicals formed in a mixture of ozone and humidified air, however, is relatively small and is highly dependent on the amount of humidity present. The addition of hydrogen peroxide to the air in which the reaction occurs can greatly increase the number of hydroxyl radicals formed. This additional hydrogen peroxide reacts with ozone, converting the ozone and hydrogen peroxide molecules to the more highly oxidative hydroxyl radicals (.OH). The resultant mixture then has two methods of eliminating the targeted pathogens, allergens and/or odor-causing agents: (i) direct oxidation by ozone; and (ii) indirect oxidation by hydroxyl radicals. These oxidation reactions generally compete for contaminants to oxidize. The ratio of direct oxidation with molecular ozone is relatively slow (10⁵−10⁷ M⁻¹sec⁻¹) compared to oxidation by hydroxyl radicals (10¹²−10¹⁴ M⁻¹sec⁻¹).

According to particularly preferred embodiments of the present invention, the concentration of hydrogen peroxide is generally at least 0.025 ppm (parts per million). Preferably, the concentration of hydrogen peroxide is at least 0.026 ppm, more preferably at least 0.027 ppm, even more preferably at least 0.028 ppm and still even more preferably at least 0.029 ppm. Most preferably, the concentration of hydrogen peroxide is at least 0.030 ppm.

According to certainly preferred embodiments of the present invention, the concentration of hydrogen peroxide is between 0.025 and 0.045 ppm. According to such embodiments, the concentration of hydrogen peroxide is preferably between 0.025 and 0.040 ppm, more preferably between 0.027 and 0.038 ppm and even more preferably between 0.030 and 0.035 ppm. Other preferred embodiments include embodiments where the concentration of hydrogen peroxide is between 0.025 ppm and 0.035 ppm and more preferably between 0.027 and 0.033 ppm. Still other preferred embodiments include embodiments where the concentrations of hydrogen peroxide is between 0.030 and 0.040 ppm, preferably between 0.033 ppm and 0.040 ppm and more preferbably between 0.035 ppm and 0.040 ppm.

According to the methods of the present invention, it is preferable to maintain the ratio of hydroxyl radicals to ozone as high as possible. Thus, preferred embodiments of the present invention include those methods where the ratio of hydrogen peroxide to ozone is at least 3:1. Preferably, the ratio of hydrogen peroxide to ozone is at least 3.2:1, more preferably at least 3.5:1, even more preferably at least 3.7:1, still even more preferably at least 3.9:1 and most preferably at least 4:1.

The methods of the present invention can be used to control or reduce the level of any pathogen, allergen and/or odor-causing agent that can be contacted, with the inventive atmosphere of ozone and hydrogen peroxide vapor. The inventive atmosphere is simply allowed to come into contact with the targeted pathogen(s), allergen(s) and/or odor-causing agents for a period of time sufficient to control or reduce the level thereof

The methods of the present invention may be used to control or reduce the level of any pathogen, allergen and/or odor-causing agent in a defined space, such as a room or house, simply by introducing the inventive atmosphere into the environment being treated.

The methods may also be used to control or reduce the level of any pathogen, allergen and/or odor-causing agent on a surface or an object, by directing a stream of the inventive atmosphere onto the surface or object being treated. Moreover, in the case of an object to be treated, such an object can be placed in a controlled space, such as a box or sealed chamber, and the inventive atmosphere introduced into the enclosed space.

The methods of the present invention can be employed to control or reduce the level of any of the substances discussed herein, but the methods are particularly preferred for pathogens, allergens, and Volatile Organic Compounds (VOCs).

Pathogens that can be controlled using the methods of the present invention include, but are not limited to, the following: Bacillus anthracis (anthrax); Clostridium botulinum (botulism); Brucella species (brucellosis); Burkholderia mallei (glanders); Burkholderia pseudomallei (melioidosis); Chlamydia psittaci (psittacosis); Coxiella burnetii (Q fever); Cryptosporidium parvum; E. coli strains, including O157:H7; emerging infectious diseases, such as Nipah virus and hantavirus; Norwalk virus; Severe Acute Respiratory Syndrome (SARS); Acquired Immune Deficiency Syndrome (AIDS) virus; Human Immunodeficiency Virus (HIV); Francisella tularensis (tularemia); Rickettsia prowazekii (typhus fever); Salmonella species (salmonellosis); Salmonella Typhi (typhoid fever); Shigella (shigellosis); Staphylococcal enterotoxin B; Variola major (smallpox); Vibrio cholerae (cholera); Viral encephalitis (including Venezuelan equine encephalitis, eastern equine encephalitis, western equine encephalitis); Viral hemorrhagic fevers (filoviruses [e.g., Ebola, Marburg] and arenaviruses [e.g., Lassa, Machupo]); and Yersinia pestis (plague).

Other pathogens that can be controlled include molds, such as Acremonium; Alternaria; Aspergillus fumigatus; Aspergillus niger; Aspergillus species Var. 1; Aspergillus species Var. 2; Aureobasidium; Bipolaris, Chaetomium; Cladosporium, Curvularia; Epicoccum; Fusarium; Geotrichum; Memnoniella; Mucor; Mycelia sterilia; Nigrospora; Paecilomyces; Penicillium species Var. 1; Penicillium species Var. 2; Pithomyces; Rhizopus; Sporothrix; Sporotrichum; Stachybotrys; Syncephalastrum; Trichoderma; and Yeast.

Indoor allergens that can be remediated by methods of preferred embodiments include dust mite feces, dander, rodent urine and cockroach allergens.

Dust mite feces are the major source of allergic reaction to household dust. The mites thrive on shed human skin and are most commonly found in bedrooms, where skin cells are abundant. Preventive measures include frequently laundering bed linens in hot water and removing carpets from the room. In some extreme cases, homeowners have even been forced to encase the bed mattress, box springs, and pillows in vinyl covers.

Other allergens of animal origin include skin scales shed from humans and animals, otherwise known as dander. Dander from such animals as cats, dogs, horses, and cows can infest a home even if the animal has never been inside.

Rodent urine from mice, rats, and guinea pigs are another group of allergens.

Cockroach-derived allergens come from the insect's discarded skins which, as they disintegrate over time, become airborne and are inhaled.

In addition, tobacco smoke, engine exhaust, and similar odor-causing agents can also be reduced or controlled by methods of the present invention, as can volatile organic compounds from sources such as household products including paints, carpets, paint strippers, and other solvents; wood preservatives; aerosol sprays; cleansers and disinfectants; moth repellents and air fresheners; stored fuels and automotive products; hobby supplies; dry-cleaned clothing, and the like. VOCs include organic solvents, certain paint additives, aerosol spray can propellants, fuels (such as gasoline, and kerosene), petroleum distillates, dry cleaning products, and many other industrial and consumer products ranging from office supplies to building materials. VOCs are also naturally emitted by a number of plants and trees. Some of the more common VOCs include ammonia, ethyl acetate, methyl propyl ketone, acetic acid, ethyl alcohol, methylene chloride, acetone, ethyl chloride, n-propyl chloride, acetylene, ethyl cyanide, nitroethane, amyl alcohol, ethyl formate, nitromethane, benzene, ethyl propionate, pentylamine, butane, ethylene, pentylene, butyl alcohol, ethylene oxide, propane, butyl formate, formaldehyde, propionaldehyde, butylamine, formic acid, propyl alcohol, butylene, heptane, isopropyl chloride, carbon tetrachloride, hexane, propyl cyanide, chlorobenzene, isobutane, propyl formate, carbon monoxide, hexyl alcohol, propylamine, chlorocyclohexane, hydrogen gas, propylene, chloroform, hydrogen sulfide, tertiary butyl alcohol, cyclohexane, isopropyl acetate, tetrachloroethylene, cylohexene, methane, toluene, 1-dichloroethane, methyl alcohol, 1,1,2-trichloroethane, 1,2-dichloroethane, methyl chloride, trichlorethylene, diethyl ketone, methyl chloroform, triethylamine, diethylamine, methyl cyanide, xylene, ethane, and methyl ethyl ketone.

Other odor-causing agents that can be reduced or controlled include skunk odors, urine, pet odors, and the like.

It is generally preferred to subject the substance to be controlled or reduced to the inventive gaseous atmosphere under conditions sufficient to provide an effective concentration of ozone and an effective concentration of hydrogen peroxide. The length of treatment can then be set or adjusted as necessary to ensure satisfactory kill and/or neutralization levels.

As noted above, any interior or contained space is amenable to treatment by methods of the present invention. For example, single family homes, apartment buildings, office buildings, schools, hospitals, doctor's offices, laboratories, restaurants, ships, trains, buses, airplanes, trucks and the like are particularly well-suited to treatment.

The methods of the present invention can also be employed to treat articles of manufacture. Articles of manufacture that can be treated include any materials that can tolerate exposure to effective concentrations of ozone and hydrogen peroxide, preferably at the humidity and temperature conditions of preferred embodiments, without suffering unacceptable damage. For example, clothing, bedding and linens, rugs, mail, packages, documents, furniture, food items, agricultural products such as seeds, grains, cut flowers, produce, fruits vegetables, and live plants, containers and packaging materials, and the like.

A suitable chamber can be constructed that can be sealed to maintain the desired conditions of ozone and hydrogen peroxide concentration, and optionally humidity and temperature levels. The articles to be treated are then placed inside that chamber and subjected to an inventive atmosphere.

In automated processes, articles to be treated can be moved through an airlock and into the chamber for treatment for a suitable time period, and then moved out of the chamber through the same or a different airlock. Such automated processes can be particularly well suited for the decontamination of medical instruments or the decontamination of animal carcasses or meat products (beef, pork, poultry, seafood, and the like) for pathogens such as salmonella and E. coli.

In other embodiments, it can be preferable to subject a room or space to periodic decontamination, such as a surgical suite in a hospital, a treatment or waiting room in a clinic, a kitchen or a restaurant, a meat processing area of a grocery store or the like. In such embodiments, it is generally preferred to permanently install equipment in a location adjacent to the space to be treated.

Any suitable method or apparatus, or combination thereof, can be used to generate ozone and hydrogen peroxide for use in the inventive methods. Particularly preferred is equipment that delivers substantially pure or pure ozone.

Commercially available devices that generate ozone and hydrogen peroxide by either ultraviolet or corona discharge are generally suitable for use in the preferred embodiments of the present invention. Illustrative examples of known devices include, but are not limited to, the devices disclosed in U.S. Pat. No. 6,955,751; U.S. Patent Publication No. 2007/0245938; and U.S. Patent Publication No. 2009/041617.

Before use in the methods of the present invention, however, such known devices must be modified so that the environment in which hydrogen peroxide is generated has sufficient relative humidity. That is, even though these devices generate hydrogen peroxide through the reaction of ozone and water vapor, the level of water vapor (i.e. relative humidity) available is generally insufficient to achieve the results of the present invention. Rather, the relative humidity within such devices much be increased by the introduction of additional water vapor.

Any suitable systems and techniques known and available to those skilled in the art may be used to ensure that the environment in which hydrogen peroxide is generated has sufficient relative humidity. For example, the output of a commercially available humidifier may directed, e.g. by a hose or tube or the like, into the environment within the device where ozone and hydrogen peroxide are generated. Alternatively, the device may be placed with a sealed, enclosed space having the requisite relative humidity.

According to the methods of the present invention, the relative humidity of the environment in which hydrogen peroxide is generated must be at least 70%. Preferably, the relative humidity is at least 75%, more preferably at least 80%, even more preferably at least 85%, still even more preferably at least 90% and yet even more preferably at least 95%. According to certain embodiments of the present invention, the relative humidity is at least 99%.

According to certain preferred embodiments of the present invention, the relative humidity is between 75% and 100%, preferably between 80% and 100%, more preferably between 85% and 100% and most preferably between 90% and 100%. According to other preferred embodiments of the inventive methods, the relative humidity is between 75% and 95%, more preferably between 80% and 95% and most preferably between 90% and 95%. According to still other preferred embodiments of the inventive methods, the relative humidity is between 80% and 95%, more preferably between 85% and 95% and most preferably between 90% and 95%. According to yet other preferred embodiments of the inventive methods, the relative humidity is between 85% and 95%. According to additional preferred embodiments, the relative humidity is between 75% and 90%, more preferably between 80% and 90% and most preferably between 85% and 90%.

According to the methods of the present invention, a substance to be treated is exposed to a gaseous atmosphere of ozone and hydrogen peroxide as described herein for a sufficient period of time to control or reduce the level of a pathogen, allergen and/or odor-causing agent. Following such treatment, it is generally preferably to remove or destroy, any residual ozone.

Any suitable method can be employed for destroying or removing lingering ozone. For example, ultraviolet (UV) light may preferably be employed. A UV light source can be brought into proximity to the substance that has been or is being treated using the inventive methods, and the light therefrom passed over the treated substance after treatment ceases. Removal of ozone can also be accelerated by subjecting the interior spaces to elevated temperatures, for example, by a radiant heater or hot air blower.

The foregoing description and examples are illustrative only and are not intended to limit the scope of the invention as defined by the appended claims. It will be apparent to those skilled in the art that various modifications and variations can be made in the kits of the present invention without departing from the spirit and scope of the invention. Thus, it is intended that the present invention cover the modifications and variations of this invention provided they come within the scope of the appended claims and their equivalents.

What is claimed is:

1. A method for controlling or reducing the level of a substance selected from the group consisting of a pathogen, an allergen and an odor-causing agent, comprising:
exposing said substance to a gaseous atmosphere comprising an effective amount of ozone and an effective amount of hydrogen peroxide, wherein said gaseous atmosphere in generated in an atmosphere having a relative humidity of at least 70% and further wherein said substance is exposed to the atmosphere for a time period sufficient to control or reduce the level of said substance.

2. The method according to claim 1, wherein said relative humidity is at least 75%.

3. The method according to claim 1, wherein said relative humidity is at least 80%.

4. The method according to claim 1, wherein said relative humidity is at least 90%.

5. The method according to claim 1, wherein said relative humidity is at least 99%.

6. The method according to claim 1, wherein said effective concentration of ozone is between 0 and 0.0070 parts per million.

7. The method according to claim 1, wherein said effective concentration of ozone is between 0 and 0.0065 parts per million.

8. The method according to claim 1, wherein said effective concentration of ozone is between 0 and 0.0060 parts per million.

9. The method according to claim 1, wherein said effective concentration of hydrogen peroxide is at least 0.025 parts per million.

10. The method according to claim 1, wherein said effective concentration of hydrogen peroxide is at least 0.030 parts per million.

11. The method according to claim 1, wherein said effective concentration of hydrogen peroxide is at least 0.034 parts per million.

12. The method according to claim 1, wherein the ratio of said effective concentration of hydrogen peroxide to said effective concentration of ozone is at least 3:1.

13. The method according to claim 1, wherein the ratio of said effective concentration of hydrogen peroxide to said effective concentration of ozone is at least 3.5:1.

14. The method according to claim 1, wherein the ratio of said effective concentration of hydrogen peroxide to said effective concentration of ozone is at least 4:1.

15. The method according to claim 1, wherein said substance is a pathogen.

16. The method according to claim 15, wherein said pathogen is selected from the group consisting of Bacillus anthracis (anthrax), Clostridium botulinum (botulism), Brucella species (brucellosis), Burkholderia mallei (glanders), Burkholderia pseudomallei (melioidosis), Chlamydia psittaci (psittacosis), Coxiella burnetii (Q fever), Cryptosporidium parvum, E. coli, Nipah virus, hantavirus, Norwalk virus, Severe Acute Respiratory Syndrome (SARS), Acquired Immune Deficiency Syndrome (AIDS) virus, Human Immunodeficiency Virus (HIV), Francisella tularensis (tularemia), Rickettsia prowazekii (typhus fever), Salmonella species (salmonellosis), Salmonella Typhi (typhoid fever), Shigella (shigellosis), Staphylococcal enterotoxin B, Variola major (smallpox), Vibrio cholerae (cholera), Viral encephalitis, Viral hemorrhagic fevers, Yersinia pestis (plague), Acremonium, Alternaria, Aspergillus fumigatus, Aspergillus niger, Aspergillus species Var. 1, Aspergillus species Var. 2, Aureobasidium, Bipolaris, Chaetomium, Cladosporium, Curvularia, Epicoccum, Fusarium, Geotrichum, Memnoniella, Mucor, Mycelia sterilia, Nigrospora, Paecilomyces, Penicillium species Var. 1, Penicillium species Var. 2, Pithomyces, Rhizopus, Sporothrix, Sporotrichum, Stachybotrys, Syncephalastrum, Trichoderma, and Yeast.

17. The method according to claim 1, wherein said substance is an allergen.

18. The method according to claim 17, wherein said allergen is selected from the group consisting of dust mites, dander, rodent urine and cockroach skins.

19. The method according to claim 1, wherein said substance is an odor-causing agent.

20. The method according to claim 19, wherein said odor-causing is selected from the group consisting of tobacco smoke, engine exhaust and volatile organic compounds.
